(19) United States
(12) Patent Application Publication
(10) Pub. No.: US 2015/0080355 A1
(43) Pub. Date: Mar. 19, 2015

(54) Pharmaceutical Compositions Comprising Ebastine and Fluticasone

(71) Applicants: Geena Malhotra, Mumbai (IN); Shrinivas Madhukar Purandare, Mumbai (IN)

(72) Inventors: Geena Malhotra, Mumbai (IN); Shrinivas Madhukar Purandare, Mumbai (IN)

(73) Assignee: CIPLA LIMITED, Mumbai (IN)

(21) Appl. No.: 14/235,874

(22) PCT Filed: Aug. 2, 2012
(86) PCT No.: PCT/GB2012/000631

§ 371 Date: Jul. 9, 2014

(30) Foreign Application Priority Data
Aug. 2, 2011 (IN) 2193/MUM/2011

(51) Int. Cl. A61K 31/56; A61K 9/00; A61K 45/06; A61K 31/4515

(52) U.S. Cl. CPC A61K 31/56; A61K 31/4515; A61K 9/0043; A61K 9/0048; A61K 45/06
USPC 514/171

(57) ABSTRACT

A pharmaceutical composition comprises at least one antihistamine, at least one corticosteroid, and at least one pharmaceutical excipient, wherein the at least one antihistamine comprises ebastine or its pharmaceutically acceptable salt, solvate, ester or physiologically functional derivative thereof, and wherein the at least one corticosteroid comprises fluticasone or its pharmaceutically acceptable ester thereof.

### FIELD OF INVENTION

The present invention relates to pharmaceutical compositions for nasal and ocular use. There is also provided a process for preparing the said compositions and their use in the treatment and/or prevention of allergic disorders.

### BACKGROUND OF INVENTION

Allergic rhinitis (AR) is an extremely common health problem, affecting approximately 10-25% of the population worldwide. Allergic rhinitis is characterized by inflammation of the upper airway mucus membranes mediated by binding of antigens to specific immunoglobulin E (IgE) antibodies. The symptoms of allergic rhinitis include congestion, runny nose postnasal drip, red itchy eyes, headaches, sneezing, pruritis of the nasal mucosa and oropharynx, allergic shiners, lacrimation, and fatigue which are most bothersome for patients.

Nasal congestion is one of the most prevalent symptoms of Allergic rhinitis and occurs in, approximately 90% of patients. In fact, nasal congestion is the symptom that is most closely associated with Allergic rhinitis related sleep problems. Other nasal and ocular symptoms such as nasal itching also play an important role in awakening patients. The effect of nasal congestion on sleep increases as the severity of congestion intensifies.

Another important aspect of Allergic rhinitis (AR) associated nasal congestion is its negative impact on the patient's quality of life.

Nasal congestion aggravates in supine position, thus worsening its effects during sleep. In addition, nasal congestion, rhinorrhoea and sneezing exhibit circadian rhythms, with the greatest intensity in the early morning, thus exacerbating their negative effects on sleep. Allergic rhinitis related inflammatory mediators also exhibit a circadian pattern, with peak levels in early morning. In addition, sympathetic tone decreases at night, resulting in a relative parasympathetic excess, which is associated with nasal congestion and reduced bronchial dilation.

The daytime tiredness experienced by the vast majority of Allergic rhinitis (AR) sufferers is directly related to the fact that patients with Allergic rhinitis experience disrupted sleep at night. In addition to this daytime fatigue and somnolence, nocturnal sleep impairment is associated with depression, irritability, memory deficits, inability to concentrate, decreased alertness and which overall leads to decreased quality of life. Consequently, many of the sequelae of Allergic rhinitis, such as fatigue, decreased cognitive functioning and work performance and reduced quality of life may be caused or worsened by Allergic rhinitis related sleep impairment.

Abnormal sleep is one such factor that classifies the severity of Allergic rhinitis from mild to moderate/severe. Thus, achievement of unimpaired sleep therefore is the primary goal of Allergic rhinitis treatment.

Allergic rhinitis associated nasal congestion results from dilation of venous capacitance vessels in the nasal sub mucosa and increased vascular permeability, mucosal oedema with influx of inflammatory cells and excess secretions. This allergic response is composed of two phases: the early phase and late phase. During the early phase, nasal allergic response antigen deposition on the mucosal surface results in binding of IgE antibodies to respiratory mucosal mast cells and peripheral blood basophils. Consequent mast cell degranulation and release of chemical mediators such as histamine, leukotrienes and pro-inflammatory cytokines are primarily responsible for sneezing, itching and rhinorrhea. Nasal congestion—the predominant late phase symptom results from the infiltration of inflammatory cells such as eosinophils and T cells into tissue and consequent prolonged release mediators such as histamine, leukotrienes and prostaglandins.

Treatment of Allergic rhinitis is commonly based on the type and severity of the individual patient's symptoms and should ideally reduce nasal congestion, sneezing and rhinorrhea over the course of entire day and night.

Antihistamines are the mainstay of therapy for Allergic rhinitis and are effective in reducing pruritus, sneezing and watery rhinorrhea. These drugs act primarily by blocking the H₁-histamine receptor. Antihistamines also interfere with mediator release from mast-cells by inhibiting either calcium ion influx across mast cell and basophil plasma membrane or intracellular calcium ion release within the cells. Further, antihistamines may also inhibit the late phase allergic reaction by acting on leukotrienes or prostagiandins or by producing an anti-platelet activating factor (PAF) effect. However, they significantly do not reduce nasal obstruction as compared to that of intranasal corticosteroids.

Corticosteroids such as intranasal and intraocular corticosteroids are considered as the first line therapy for moderate to severe seasonal and perennial Allergic rhinitis.

Corticosteroids known for intranasal use include beclomethasone, mometasone, fluticasone, budesonide and ciclesonide.

Corticosteroids known for ocular anti-inflammatory use include betamethasone sodium, dexamethasone sodium and prednisolone acetate.

Intranasal corticosteroids prevent both the early phase (cytokine release) and late phase (migration of mast cells, basophils and eosinophils to the nasal mucosa) allergic reaction. Intranasal corticosteroids also decrease microvascular permeability, edema and mucus secretion. Intranasal corticosteroids suppress many of the inflammatory mediators implicated in the allergic reaction and effectively reduce nasal symptoms including congestion, rhinorrhea, sneezing, pruritus, ocular itching, redness and tears. Onset of effect of intranasal corticosteroids occurs after 6-12 hours and maximum benefit is achieved after a week or more of regular use.

Intranasal corticosteroids are generally considered safe in adults and children due to their topical administration and low systemic bioavailability. Intranasal corticosteroids are one of the most effective agents for controlling nasal obstruction, and thus reduce sleep problems and associated daytime somnolence.

WO9746243 discloses a nasal spray composition of a safe and effective amount of a glucocorticoid such as beclomethasone, flunisolide, fluticasone, mometasone, budesonide and a safe effective amount of a fast acting antihistamine such as acrivastine, carbinoxamine, diphenhydramine, chloropheniramine, brompheniramine, dexchloropheniramine, doxylamine, clemastine, promethazine, rocastine, trimeprazine, methdilazine, hydroxyzine, pyrilamine, tripelennamine, meclizine, triprolidine, azatadine, cyproheptadine, phenindamine and an aqueous intranasal carrier and the composition is free of capsaicin.

US20090324699 discloses a liposomal delivery of a pharmaceutical composition comprising an antihistamine a corticosteroid and a pharmaceutically acceptable aqueous carrier. However, such compositions involve use of a complex process for manufacturing such liposomes.

Combination therapy of an antihistamine and an intranasal or intraocular corticosteroid provides instant relief from the allergic symptoms and controls progression of the disease. Further, selection of a specific antihistamine and an intranasal or intraocular corticosteroid plays a very important role in formulation of such combinations.

Additionally it simplifies the therapy, reduces the cost and also provides control of both early phase and late phase symptoms of Allergic rhinitis.

Hence, there still remains a need to formulate a pharmaceutical composition which is efficacious, and exhibits potent topical activity for the treatment and/or prevention of allergic disorders.

### OBJECT OF THE INVENTION

The object of the present invention is to provide a pharmaceutical composition comprising an antihistamine and a corticosteroid for administration in treatment and/or prevention of allergic disorders.

Another object of the present invention is to provide a process for preparing a pharmaceutical composition comprising an antihistamine and a corticosteroid for administration in treatment and/or prevention of allergic disorders.

Yet another object of the present invention is to provide a method for prevention and/or treatment of allergic disorders which method comprises administering a pharmaceutical composition comprising an antihistamine and a corticosteroid.

### SUMMARY OF THE INVENTION

According to one aspect of the present invention, there is provided a pharmaceutical composition comprising at least one antihistamine and at least one corticosteroid.

According to another aspect of the present invention, there is provided a process for preparing the pharmaceutical composition comprising at least one antihistamine and at least one corticosteroid.

According to yet another aspect of the invention there is provided a pharmaceutical composition comprising at least one antihistamine, at least one corticosteroid, and at least one pharmaceutically acceptable excipients, wherein at least one antihistamine comprises ebastine or its pharmaceutically acceptable salt, solvate, ester or physiologically functional derivative thereof, and wherein at least one corticosteroid comprises fluticasone or its pharmaceutically acceptable ester thereof.

According to another aspect of the invention, there is provided a pharmaceutical composition of the invention for use in treating disorders or conditions that respond to, or are prevented, ameliorated or eliminated by the administration of an antihistamine and a corticosteroid.

According to another aspect of the invention, there is provided a method for the prevention and/or treatment of a disorder or condition that responds to, is prevented, ameliorated or eliminated by the administration of an antihistamine and a corticosteroid, which method comprises administering to a patient in need thereof, a therapeutically effective amount of a pharmaceutical composition of the invention.

According to another aspect of the invention, there is provided a process for the preparation of a pharmaceutical composition according to the present invention.

### DETAILED DESCRIPTION OF THE INVENTION

As discussed earlier it is highly desirable to provide a treatment for allergic disorders that combines the effects of antihistamine and a corticosteroid, in a pharmaceutically acceptable composition which is tolerated in situ, without significantly disrupting the potency of the constituent pharmaceuticals.

Our inventors have found that antihistamine such as ebastine can advantageously be combined with a corticosteroid such as fluticasone to provide a stable and effective combination product or composition for the treatment of allergic disorders.

Ebastine is not very soluble in water and exhibits low bioavailability in the commercially available dosage forms. Further since this a nasal preparation; there is a very limited choice of excipients/solvents available.

Surprisingly, our inventors have developed a nasal dosage form wherein ebastine has been formulated in a nasal dosage form inspite of the limitations as stated above.

The terms “ebastine” and “fluticasone” are used in broad sense to include, not only “ebastine” and “fluticasone” per se, but also their pharmaceutically acceptable salts, pharmaceutically acceptable solvates, pharmaceutically acceptable esters, pharmaceutically acceptable hydrates, pharmaceutically acceptable enantiomers, pharmaceutically acceptable derivatives, pharmaceutically acceptable polymorphs, etc.

The term “derivative” includes, but is not limited to, pharmacologically active metabolites and prodrugs.

Preferably the intranasal corticosteroid used in the compositions of the present invention is fluticasone.

Any ester of fluticasone can be used in the pharmaceutical compositions of the present invention. Preferably, the esters of fluticasone are selected from the group comprising fluticasone propionate, fluticasone furoate, fluticasone valerate.

Fluticasone is currently commercially available in the form of fluticasone furoate and fluticasone propionate.

Fluticasone propionate is a synthetic trifluorinated corticosteroid having the chemical name S-(fluoromethyl) 6α,9-difluoro-11β-17-dihydroxy-16α-methyl-3-oxoandrosta-1,4-diene-17β-carbothioate, 17-propionate. Fluticasone propionate is a topically active corticosteroid with efficacy in seasonal and perennial Allergic rhinitis. Fluticasone propionate also exhibits high lipophilicity, high selectivity and affinity for the glucocorticoid receptor, low oral systemic absorption, and rapid metabolic clearance. Fluticasone furoate is a synthetic, lipophilic, trifluorinated glucocorticoid receptor agonist containing a 17-alpha-furoate ester having the chemical name (6α,11β16α,17α)-6,9-difluoro-17-{[(fluoro-methyl)thio]carbonyl}-11-hydroxy-16-methyl-3-oxoandrosta-1,4-dien-17-yl 2-furancarboxylate. Further, fluticasone furoate is a novel corticosteroid which substantially overcomes the potential side effects that are generally produced by the use of conventional corticosteroids.

Preferably the antihistamine used in compositions of the present invention is ebastine.

Ebastine, (4-diphenylmethoxy-1-[3-(4-terbutylbenzoyl)-propyl]piperidine), is a long-acting and selective H₁-histamine receptor antagonist. Ebastine is converted to the pharmacologically active acid metabolite, carebastine. Ebastine is indicated for the symptomatic treatment of seasonal and perennial allergic rhinitis and idiopathic chronic urticaria. Ebastine is commercially available as film-coated, 10 mg oral tablets and in the form of pediatric syrup. Ebastine is generally administered once daily in strengths of 10 mg, and is a nonsedating antihistamine for the treatment of symptoms associated with seasonal and perennial allergic rhinitis. Ebastine is also highly effective in the therapy and treatment of seasonal and perennial allergic rhinitis and related diseases.

However, ebastine is not very soluble in water and as a result of which it does not become readily bioavailable when given orally. Thus, it would also be desirable to formulate a more soluble and more bioavailable form of antihistamine such as ebastine. Such a formulation would be fast acting, thereby providing immediate relief to a subject suffering from rhinitis, urticaria and such related disorders much more quickly.

Thus, the present invention provides a pharmaceutical composition comprising ebastine and fluticasone for administration via nasal and ocular route.

Fluticasone is preferably present in an amount from about 10 mcg to 70 mcg, and more preferably from about 20 mcg to about 50 mcg.

Preferably, fluticasone propionate is present in the amount of about 50 mcg, and fluticasone furoate is present in the amount of about 27.5 mcg.

Ebastine may be present in the in the amount of about 25 mcg to about 2 mg.

Preferably, the pharmaceutical compositions of the invention are free of liposomes.

Preferably, the pharmaceutical compositions of the invention are free of fast acting antihistamines. Preferably, the compositions of the invention are free of antihistamines wherein the onset of action occurs within one hour of administration. Preferably, the pharmaceutical compositions of the invention are free of acrivastine, carbinoxamine, diphenylhydramine, chloropheniramine, brompheniramine, dexchloropheniramine, doxylamine, clemastine, promethazine, trimeprazine, methdilazine, hydroxyzine, pyrilamine, rocastine, tripelennamine, meclizine, triprolidine, azatadine, cyproheptadine, phenindamine, and pharmaceutically acceptable salts thereof.

The pharmaceutical compositions of the present invention may be administered by any suitable methods used for delivery of the drugs to the respiratory tract.

The pharmaceutical compositions of the invention can be formulated for simultaneous, separate or sequential administration.

The pharmaceutical composition of the present invention can be formulated in a suitable nasal dosage form.

The pharmaceutical compositions, of the present invention, preferably comprise pharmaceutically acceptable excipients suitable for nasal delivery, and the at least one antihistamine and at least corticosteroid are present in a dosage form suitable for nasal delivery.

Preferably, the pharmaceutical compositions of the present invention are formulated in a form suitable for nasal delivery such as but not limited to nasal spray, nasal solutions, nasal suspensions, nasal ointments, nasal drops and nasal gels.

The pharmaceutical composition of the present invention may comprise ebastine and fluticasone in a suitable nasal dosage form with one or more pharmaceutically acceptable excipients.

The preferred dosage forms, suitable for nasal delivery, according to the present invention may comprise carriers/excipients suitable for formulating the same such as, but not limited to, pH adjusters, osmotic agents, emulsifiers or dispersing agents, surfactants, solubilizers, buffering agents, preservatives, wetting agents, gelling agents, consistency agents, ciliary stimulant, mucus thinning agent and mixtures thereof.

The buffer or the pH adjusting agent may comprise one or more of organic or inorganic acids such as, but not limited to, citric acid, citric acid monohydrate, sodium citrate dehydrate, sodium hydrogen sulphate borate buffer, phosphates (sodium hydrogen orthophosphate, disodium hydrogen phosphate, Sodium dihydrogen phosphate), trometamol, acetate buffer, citrate buffer and their hydrates, or equivalent conventional buffers, for example, to adjust the formulations to a pH value of about 3.0 to about 7.5.

Suitable preservatives may also be employed in the pharmaceutical composition of the present invention to protect the formulation from contamination with pathogenic bacteria.

The preservative may comprise one or more of, but is not limited to, benzalkonium chloride, benzoic acid or a salt, sodium benzoate, potassium sorbate, sorbic acid or a salt, edetic acid and its alkali salts, lower alkyl p-hydroxybenzoates, chlorhexidine, phenyl mercury borate, quaternary ammonium compound or mixtures thereof.

According to the present invention, preservative is present in an amount of from about 0.005 to 2%.

Osmotic agents refer to agents that are specifically added to the composition to increase the solute level in the composition and contribute to achieving isotonicity of the pharmaceutical composition.

Tonicity is the ‘effective osmolality’ and is equal to the sum of the concentrations of the solutes which have the capacity to exert an osmotic force across the membrane.

Isotonic conditions are required for ophthalmic, nasal, most electrolyte and other preparations. Hypertonic solution will cause water to leave the intracellular compartment with consequent cell shrinkage while hypotonic solution will cause the cell to imbibe water which produces swelling, distention and finally rupture of the cells.

Osmotic agents, that may be used, comprise, but are not limited to, sodium chloride, potassium chloride, zinc chloride, calcium chloride, mannitol, glycerol, and boric acid, citric acid, sodium tartrate, sodium phosphate, potassium phosphate, propylene glycol or other inorganic or organic solutes, dextrose/anhydrous glucose and mixtures thereof.

Further as per common general knowledge known to one skilled in the art, 0.9% w/v sodium chloride solution is said to be isotonic (308 mOsm/kg) with body fluids while glycerin at 2.6% w/v concentration is iso-osmotic with 0.9% w/v saline solution.

Chelating agents according to the present invention may comprise, but are not limited to, editic acid (EDTA) or one of the known salts thereof, e.g. sodium EDTA or disodium EDTA dihydrate (sodium edetate) and mixtures thereof.

According to the present invention, Chelating agents may be present in an amount of from about 0.01 to 5%.

Suitable surfactants or wetting agents may also be used in the pharmaceutical compositions of the present invention. According to the present invention, suitable amphoteric, non-ionic, cationic or anionic surfactants may be included in the pharmaceutical composition of the present invention.

Surfactant may comprise one or more, but are not limited to, Polysorbates such as polysorbate 20, polysorbate 40, polysorbate 60, polysorbate 80, polysorbate 65, polysorbate 85, sorbitan fatty acid esters such as Span 20, Span 40, Span 60 Span 80, Span 120; sodium lauryl sulfate; polyethoxylated castor oil; polyethoxylated hydrogenated castor oil, sodium dodecyl sulfate (sodium lauryl sulfate), Lauryl dimethyl amine oxide, Docusate sodium, Cetyl trimethyl ammonium bromide (CTAB) Polyethoxylated alcohols, Polyoxyethylene sorbitan, Octoxynol, N,N-dimethyldodecylamine-N-oxide, Hexadecyltrimethylammonium bromide, Polyoxyl 10 lauryl ether, Brij, Bile salts (sodium deoxycholate, sodium cholate), Polyoxyl castor oil, Nonylphenol ethoxylate, Cyclodextrins, Lecithin, Methylbenzethonium chloride. Carboxylates, Sulphonates, Petroleum sulphonates, alkylbenzenesulphonates, Naphthalenesulphonates, Olefin sulphonates, Alkyl sulphates, Sulphates, Sulphated natural oils & fats, Sulphated esters, Sulphated alkanolamides, Alkylphenols, ethoxylated & sulphated, Ethoxylated aliphatic alcohol, polyoxyethylene surfactants, carboxylic esters Polyethylene glycol esters, Anhydrosorbitol ester & it's ethoxylated derivatives, Glycol esters of fatty acids, Carboxylic amides, Monoalkanolamine condensates, Polyoxyethylene fatty acid amides, Quaternary ammonium salts, Amines with amide linkages, Polyoxyethylene alkyl & alicyclic amines, N,N,N,N tetrakis substituted ethylenediamines 2-alkyl 1-hydroxyethyl 2-imidazolines, N-coco 3-aminopropionic acid/sodium salt, N-tallow 3-iminodipropionate disodium salt, N-carboxymethyl n dimethyl n-9 octadecenyl ammonium hydroxide, n-cocoamidethyl n-hydroxyethylglycine sodium salt etc and mixtures thereof.

Surfactants maybe present in an amount of from about 0.005 to 0.2% w/w. Suitable suspending or thickening agents may also be used in the pharmaceutical composition of the present invention, include, but are not limited to, cellulose derivatives (for example cellulose ether) in which the cellulose-hydroxy groups are partially etherified with lower unsaturated aliphatic alcohols and/or lower unsaturated aliphatic oxyalcohols (for example methyl cellulose, carboxymethyl cellulose, hydroxypropylmethylcellulose), gelatin, polyvinylpyrrolidone, tragacanth, ethoxose (water soluble binding and thickening agents on the basis of ethyl cellulose), alginic acid, polyvinyl alcohol, polyacrylic acid, pectin, microcrystalline cellulose, and equivalent agents or mixtures thereof. Should these substances contain acid groups, the corresponding physiologically acceptable salts may also be used.

According to the present invention, thickening agents can be present in an amount of from about 0.5 to 5% w/w. Consistency aids may provide enhanced physical stability as well as proper consistency to the pharmaceutical composition prior to administration so that an optimal degree of spreading over the mucosa is achieved after administration. Consistency aids may reduce or delay the rate at which the active agents in the nasal spray composition are adsorbed by the mucin of the mucosa which permits the pharmaceutical composition to have a better spread and coat the nasal mucosa.

Consistency aids that are used in compositions of the present invention may comprise, but are not limited to, low molecular weight mono and polyols selected from the group consisting of monosaccharides, disaccharides and other sugars, ribose, glycerine/glycerol, sorbitol, xylitol, inositol, propylene glycol, galactose, mannose, xylose, rhamnose, glutaraldehyde, invert sugars, ethanol, honey, mannitol, polyethylene glycol, and mixtures thereof.

Cilia along the inside of the nasal cavity keep the nasal passages clear of mucus. If cilia function is subnormal, mucus will build up and contribute to congestion of the nasal passages.

Ciliary stimulants that are used in compositions of the present invention may comprise saline solutions.

Mucus thinning agent lowers the viscosity of the mucus thereby making it more susceptible to transport by the cilia.

Mucus thinning agents that are used in compositions of the present invention may comprise alkaline agent/s but are not limited to sodium bicarbonate.

Suitable moisturizers and lubricants can also be incorporated in the pharmaceutical composition of the present invention such as, but not limited to, glycerin which prevents cracking and scaling within the inside of the nose.

The pharmaceutical composition according to the present invention is dispensed in suitable containers provided with means for enabling the application of the pharmaceutical composition to the respiratory tract.

For example, pharmaceutical composition can be administrated into the nasal passages of a subject by means of a dropper (or pipette) that includes a glass, plastic or metal dispensing tube. Fine droplets and sprays can be provided by an intranasal pump dispenser or squeeze bottle as well known in the art.

According to a preferred embodiment, the pharmaceutical composition may be present in a container comprising an elongated discharge member formed for insertion into a nasal cavity. A reservoir is coupled to the discharge member with spray actuation being achieved by squeezing the discharge member towards the reservoir.

The pharmaceutical composition, according to the present invention, may be applied to the nasal mucosa from about once a day to about three times a day and may also vary depending upon individual needs.

Preferably, the pharmaceutical composition of the invention when formulated in a way suitable for nasal administration, comprises at least one or more of a surfactant, a thickening agent, an osmotic agent, a preservative, a chelating agent or water.

Even more preferably, the pharmaceutical composition of the invention when formulated in a way suitable for nasal administration, comprises at least one or more of dispersible cellulose, polysorbate 80, anhydrous glucose, benzalkonium chloride, phenyl ethyl alcohol, disodium edetate, or purified water.

Preferably, the compositions comprise a surfactant in an amount of from about 0.005 to 0/1% w/w.

Preferably, the compositions comprise a thickener in an amount of from about 1.5 to 2% w/w.

Preferably, the compositions comprise an osmotic agent in an amount of from about 3 to 6%.

Preferably, the compositions comprise preservatives in an amount of from about 0.01 to 0.4% of preservatives.

Preferably, the compositions comprise a chelating agent in an amount of from 0.01 to 5% w/w.

Preferably, the composition comprises from 0.005 to 0.1% w/w of a surfactant, from 1.5 to 2% w/w of a thickener, from 3 to 6% of anhydrous glucose, from 0.01 to 0.4% w/w of preservatives, and/or from 0.01 to 5% w/w of a chelating agent.

In another embodiment the pharmaceutical composition of the present invention can be formulated in a suitable ocular dosage form.

The composition of the present invention can thus be administered as but not limited to, ophthalmic drops, suspension, solution, gel, ointment, in situ gel, occusert, emulsion.

The pharmaceutical composition of the present invention may comprise ebastine and fluticasone in a suitable ocular dosage form with one or more pharmaceutically acceptable excipients such as but not limited to such as tonicity-adjusting agents, pH-adjusting agents, buffering agents, preservatives, comfort enhancing agents, viscosity modifying agents, stabilizing agents, antioxidants, wetting and spreading agents.

The pharmaceutical composition, according to the present invention, may be applied to the eyes from about once a day to a few times a day and may also vary depending upon individual needs.

The pharmaceutical composition of the present invention can also be delivered by the use of other means such as, but not limited to, nasal spray, metered dose inhalers (MDI), dry powder inhalers (DPI), nebuliser, and insufflation powders.

Several types of MDIs are regularly used for administration by inhalation. These types of devices comprise but are not limited to breath-actuated MDI, dry powder inhaler (DPI), spacer/holding chambers in combination with MDI, and nebulizers.

The metered dose inhalers, according to the present invention may comprise one or more pharmaceutically acceptable excipients as HFC/HFA propellants, co-solvents, bulking agents, non volatile component, buffers/pH adjusting agents, surface active agents, preservatives, chelating agents, or combinations thereof.

Propellants are those which, when mixed with the cosolvent(s), form a homogeneous propellant system in which a therapeutically effective amount of the medicament can be dissolved. The HFC/HFA propellant must be toxicologically safe and must have a vapor pressure which is suitable to enable the medicament to be administered via a pressurized MDI.

According to the present invention the HFC/HFA propellants may comprise, one or more, but not limited to, 1,1,1,2-tetrafluoroethane (HFA-134(a)) and 1,1,1,2,3,3,3,-heptafluoropropane (HFA-227), HFC-32 (difluoromethane), HFC-143(a) (1,1,1-trifluoroethane), HFC-134 (1,1,2,2-tetrafluoroethane), and HFC-152a (1,1-difluoroethane) and such other propellants which may be known to the person having a skill in the art and mixtures thereof.

Co-solvent is any solvent which is miscible in the formulation in the amount desired and which, when added provides a formulation in which the medicament can be dissolved. The function of the cosolvent is to increase the solubility of the medicament and the excipients in the formulation.

According to the present invention the co-solvent may comprise one or more of, C₂-C₆ aliphatic alcohols, such as, but not limited to, ethyl alcohol and isopropyl alcohol; glycols such as but not limited to propylene glycol, polyethylene glycols, polypropylene glycols, glycol ethers, and block copolymers of oxyethylene and oxypropylene; and other substances, such as, but not limited to, glycerol, polyoxyethylene alcohols, and polyoxyethylene fatty acid esters; hydrocarbons such as, but not limited to, n-propane, n-butane, isobutane, n-pentane, iso-pentane, neo-pentane, and n-hexane; and ethers such as, but not limited to, diethyl ether or mixtures thereof.

Suitable surfactants may be employed in the aerosol solution composition of the present invention which may serve to stabilize the solution formulation and improve the performance of valve systems of the metered dose inhaler.

According to the present invention the surfactant may comprise one or more ionic and/or non-ionic surfactant, such as, but not limited to oleic acid, sorbitan trioleate, lecithin, isopropylmyristate, tyloxapol, polyvinylpyrrolidone, polysorbates such as polysorbate 80, vitamin E-TPGS, and macrogol hydroxystearates such as macrogol-15-hydroxystearate or mixtures thereof.

Non-volatile component is all the suspended or dissolved constituents that would be left after evaporation of the solvent.

According to the present invention, the non-volatile component may comprise one or more of monosaccharides such as, but not limited to, glucose, arabinose; disaccharides such as lactose, maltose; oligosaccharides and polysaccharides such as but not limited to dextrans; polyalcohol such as but not limited to glycerol, sorbitol, mannitol, xylitol; salts such as but not limited to potassium chloride, magnesium chloride, magnesium sulphate, sodium chloride, sodium citrate, sodium phosphate, sodium hydrogen phosphate, sodium hydrogen carbonate, potassium citrate, potassium phosphate, potassium hydrogen phosphate, potassium hydrogen carbonate, calcium carbonate and calcium chloride or mixtures thereof.

Suitable bulking agents may be employed in metered dose inhalation composition of the present invention.

According to the present invention, the bulking agent may comprise one or more, but not limited to, saccharides, including monosaccharides, disaccharides, polysaccharides and sugar alcohols such as arabinose, glucose, fructose, ribose, mannose, sucrose, terhalose, lactose, maltose, starches, dextran or mannitol or mixtures thereof.

Suitable buffers or pH adjusting agents may be employed in the metered dose inhalation composition of the present invention.

The buffer or the pH adjusting agent may comprise one or more of, but not limited to, organic or inorganic acids such as but not limited to citric acid, ascorbic acid, hydrochloric acid, sulfuric acid, nitric acid, or phosphoric acid or mixtures thereof.

Suitable preservatives may be employed in the aerosol solution composition of the present invention to protect the formulation from contamination with pathogenic bacteria.

The preservative may comprise one or more, but not limited to, benzalkonium chloride, benzoic acid, benzoates such as sodium benzoate and such other preservatives which may be known to the person having a skill in the art or mixtures thereof.

Suitable chelating agents may be employed in the aerosol solution composition of the present invention which is capable of forming complex bonds.

The chelating agent may comprise one or more but not limited to, sodium EDTA or disodium EDTA or mixtures thereof.

The pharmaceutical composition of the present invention may be administered by a dry powder inhaler (DPI).

The pharmaceutically acceptable excipients suitable for dry powder inhalation according to the present invention may be selected from suitable carriers which include, but are not limited to, sugars such as glucose, saccharose, lactose and fructose, starches or starch derivatives, oligosaccharides such as dextrins, cyclodextrins and their derivatives, polyvinylpyrrolidone, alginic acid, tylose, silicic acid, cellulose, cellulose derivatives (for example cellulose ether), sugar alcohols such as mannitol or sorbitol, calcium carbonate, calcium phosphate, etc. lactose, lactitol, dextrates, dextrose, maltodextrin, saccharides including monosaccharides, disaccharides, polysaccharides; sugar alcohols such as arabinose, ribose, mannose, sucrose, trehalose, maltose, dextran or mixtures thereof.

The pharmaceutical composition of the present invention may be administered by nebulizer. Such nebulizers include, but are not limited to, a jet nebulizer, ultrasonic nebulizer and breath actuated nebulizer. Preferably, the nebulizer is a jet nebulizer connected to an air compressor with adequate air flow. The nebulizer being equipped with a mouthpiece or suitable face mask. Specifically, a nebulizer (with face mask or mouthpiece) connected to a compressor may be used to deliver the inhalation liquid of the present invention to a patient.

Nebulisation therapy has an advantage over other inhalation therapy, since it is easy to use and does not require co-ordination or much effort. It also works much more rapidly than medicines taken by mouth.

For nebulizers, the composition according to the present invention may comprise suitable excipients such as tonicity agents, pH regulators, chelating agents in a suitable vehicle.

Isotonicity-adjusting agents, which may be used, comprise sodium chloride, potassium chloride, zinc chloride, calcium chloride and mixtures thereof. Other isotonicity-adjusting agents may also include, but are not limited to, mannitol, glycerol, and dextrose and mixtures thereof.

The pH may be adjusted by the addition of pharmacologically acceptable acids. Pharmacologically acceptable inorganic acids or organic acids may be used for this purpose. Examples of preferred inorganic acids are selected from the group comprising hydrochloric acid, hydrobromic acid, nitric acid, sulphuric acid and phosphoric acid. Examples of particularly suitable organic acids are selected from the group comprising ascorbic acid, citric acid, malic acid, tartaric acid, maleic acid, succinic acid, fumaric acid, acetic acid, formic acid and propionic acid or mixtures thereof.

Chelating agents, that may be used, according to the present invention may comprise editic acid (EDTA) or one of the known salts thereof, e.g. sodium EDTA or disodium EDTA dihydrate (sodium edetate) or mixtures thereof,

Anti-microbial preservative agent may be added for multi-dose packages.

The composition according to the present invention may be included in suitable containers provided with means enabling the application of the pharmaceutical composition to the respiratory tract. When the compositions of the present invention are formulated for nasal or ocular delivery, the containers are preferably adapted for introduction of the pharmaceutical composition to the nasal passages or eyes.

The powder for inhalation intended to be used for DPI may either be encapsulated in capsules of gelatin or HPMC or in blisters or alternatively, the dry powder may be contained as a reservoir either in a single dose or multi-dose dry powder inhalation device.

Alternatively, the powder for inhalation intended to be used for DPI may be suspended in a suitable liquid vehicle and packed in an aerosol container along with suitable propellants or mixtures thereof.

Further, the powder for inhalation intended to be used for DPI may also be dispersed in a suitable gas stream to form an aerosol composition.

The MDI composition according to the present invention may be packed in plain aluminium cans or SS (stainless steel) cans. Some aerosol drugs tend to adhere to the inner surfaces, i.e., walls of the cans and valves, of the MDI. This can lead to the patient getting significantly less than the prescribed amount of the active agent upon each activation of the MDI. Coating the inner surface of the container with a suitable polymer can reduce this adhesion problem. Suitable coatings include fluorocarbon copolymers such as FEP-PES (fluorinated ethylene propylene and polyethersulphone) and PFA-PES (perfluoroalkoxyalkane and polyethersulphone), epoxy and ethylene. Alternatively, the inner surfaces of the cans may be anodized, plasma treated or plasma coated.

The pharmaceutical compositions of the present invention may also comprise the actives in micronized form.

Poorly water-soluble drugs often require high doses in order to reach therapeutic plasma concentrations after oral administration. Improvement in the extent and rate of dissolution is highly desirable for such compounds, as this can lead to an increased and more reproducible oral bioavailability and subsequently to clinically relevant dose reduction and more reliable therapy.

Physical modifications of the drug particles such as micronization aim to increase the surface area, solubility and/or wettability of the powder particles, micronization is used to raise drug activity by increasing particle specific surface, or by allowing active substances to reach their site of action by reducing particle size.

The actives in micronized form can be obtained by any of the process such as but not limited to ball milling, jet milling, sonication, homogenization and solvent precipitation.

The pharmaceutical compositions of the present invention may also comprise the actives in nanosize form.

Nanonization of hydrophobic or poorly water-soluble drugs generally involves the production of drug nanocrystals through either chemical precipitation (bottom-up technology) or disintegration (top-down technology). Different methods may be utilized to reduce the particle size of the hydrophobic or poorly water soluble drugs. [Huabing Chen et al., discusses the various methods to develop nanoformulations in “Nanonization strategies for poorly water-soluble drugs,” Drug Discovery Today, Volume 00, Number 00, March 2010].

Nanosizing leads to increase in the exposure of surface area of particles leading to an increase in the rate of dissolution.

The nanoparticles of the present invention can be obtained by any of the process such as but not limited to milling, precipitation and homogenization.

Accordingly, the process of milling comprises dispersing drug particles in a liquid dispersion medium in which the drug is poorly soluble, followed by applying mechanical means in the presence of grinding media to reduce the particle size of drug to the desired effective average particle size.

Accordingly, the process of precipitation involves the formation of crystalline or semi-crystalline drug nanoparticles by nucleation and the growth of drug crystals. In a typical procedure, drug molecules are first dissolved in an appropriate organic solvent such as acetone, tetrahydrofuran or N-methyl-2-pyrrolidone at a super saturation concentration to allow for the nucleation of drug seeds. Drug nanocrystals are then formed by adding the organic mixture to an antisolvent like water in the presence of stabilizers such surfactants.

The choice of solvents and stabilizers and the mixing process are key factors to control the size and stability of the drug nanocrystals.

Accordingly, the process of homogenization involves passing a suspension of crystalline drug and stabilizers through the narrow gap of a homogenizer at high pressure (500-2000 bar). The pressure creates powerful disruptive forces such as cavitation, collision and shearing, which disintegrate coarse particles to nanoparticles.

Accordingly, the process of high pressure homogenization comprises drug presuspension (containing drug in the micrometer range) by subjecting the drug to air jet milling in the presence of an aqueous surfactant solution. The presuspension is then subjected to high-pressure homogenization in which it passes through a very small homogenizer gap of ˜25 μm which leads to a high streaming velocity. High-pressure homogenization is based on the principle of cavitations (i.e., the formation, growth, and implosive collapse of vapor bubbles in a liquid).

Accordingly, the process of spray-freeze drying involves the atomization of an aqueous drug solution into a spray chamber filled with a cryogenic liquid (liquid nitrogen) or halocarbon refrigerant such as chlorofluorocarbon or fluorocarbon. The water is removed by sublimation after the liquid droplets solidify.

Accordingly, the process of supercritical fluid technology involves controlled crystallization of drug from dispersion in supercritical fluids, carbon dioxide.

Accordingly, the process of double emulsion/solvent evaporation technique involves preparation of oil/water (o/w) emulsions with subsequent removal of the oil phase through evaporation. The emulsions are prepared by emulsifying the organic phase containing drug, polymer and organic solvent in an aqueous solution containing emulsifier. The organic solvent diffuses out of the polymer phase and into the aqueous phase, and is then evaporated, forming drug-loaded polymeric nanoparticles.

Accordingly, the process of PRINT (Particle replication in non-wetting templates) involves utilization of a low surface energy fluoropolymeric mold that enables high-resolution imprint lithography, to fabricate a variety of organic particles. PRINT can precisely manipulate particle size of drug ranging from 20 nm to more than 100 nm.

Accordingly, the process of thermal condensation involves use of capillary aerosol generator (CAG) to produce high concentration condensation submicron to micron sized aerosols from drug solutions.

Accordingly, the process of ultrasonication involves application of ultrasound during particle synthesis or precipitation, which leads to smaller particles of drug and increased size uniformity.

Accordingly, the process of spray drying involves supplying the feed solution at room temperature and pumping it through the nozzle where it is atomized by the nozzle gas. The atomized solution is then dried by preheated drying gas in a special chamber to remove water moisture from the system, thus forming dry particles of drug.

The pharmaceutical compositions of the invention can be manufactured by any of the types of processes as described above.

It may be well acknowledged to a person skilled in the art that the said pharmaceutical composition, according to the present invention, may further comprise one or more active(s), but not limited to and selected from anticholinergics, antiallergics, leukotriene antagonist, decongestants, sympathomimetic agents, mucolytics, opiate analgesics, lipoxygenase inhibiting compounds or their pharmaceutically acceptable salts, solvates, tautomers, derivatives, enantiomers, isomers, hydrates, prodrugs or polymorphs thereof.

The present invention also provides a process/s to manufacture the compositions according to the present invention.

The present invention provides a process of preparing an inhalation liquid which process comprises dissolving the drugs, optionally chelating agents, osmotic/isotonicity adjusting agents and any other suitable ingredients in the vehicle and adjusting the pH using a suitable pH adjusting agent.

There is also provided a process of preparing a metered dose inhalation composition which process comprises admixing a pharmaceutically acceptable carrier or excipient with the actives and the propellant and providing the composition in precrimped cans.

There is also provided a process of preparing a dry powder inhalation composition which process comprises admixing of a pharmaceutically acceptable carrier or excipient with the actives and providing the composition suitable to be administered as a dry powder inhaler with a suitable device.

The present invention also provides a method for the treatment and/or prevention of allergic disorders, which method comprises administration of a therapeutically effective amount of a pharmaceutical composition according to the present invention.

The present invention further provides a method for the treatment of nasal polyps, which method comprises administration of a therapeutically effective amount of a pharmaceutical composition according to the present invention.

The present invention further provides a method for the treatment of urticaria, which method comprises administration of a therapeutically effective amount of a pharmaceutical composition according to the present invention.

The present invention also provides a use of the pharmaceutical composition according to the present invention, in the manufacture of a medicament for the treatment of nasal polyps.

The present invention also provides a use of the pharmaceutical composition according to the present invention, in the manufacture of a medicament for the treatment of urticaria.

The present invention provides a pharmaceutical composition comprising at least one antihistamine and at least one corticosteroid for use in treating and preventing disorders or conditions that respond to, or are prevented, ameliorated or eliminated by, the administration of at least one antihistamine and at least one corticosteroid.

The present invention preferably relates to methods for the treatment and/or prevention of allergic disorders, characterized in that ebastine and fluticasone are administered in therapeutically effective amounts.

The following examples are for the purpose of illustration of the invention only and are not intended in any way to limit the scope of the present invention.

### Example 1

| Sr. No. | Ingredients | Qty | |
|---|---|---|---|
| 1. | Ebastine | 50 | mcg/spray |
| 2. | Fluticasone Propionate | 50 | mcg/spray |
| 3. | Polysorbate 80 | 0.005- 0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |
| 8. | Disodium edetate | 0.01-0.5% | w/w |

| | | |
|---|---|---|
| 9. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Propionate and Ebastine were added to the solution obtained in step (3) to produce uniform slurry.
5) The slurry obtained in step (4) was added to the main bulk obtained in step (2) and homogenized.
6) Benzalkonium chloride, Phenyl ethyl alcohol and Disodium edetate were added to the main bulk obtained in step (2).
7) Weight was made up with Purified water

### Example 2

| Sr. No. | Ingredients | Qty | |
|---|---|---|---|
| 1. | Ebastine | 50 | mcg/spray |
| 2. | Fluticasone Propionate | 50 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |

| | | |
|---|---|---|
| 8. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Propionate and Ebastine were added to the solution obtained in step (3) to produce uniform slurry.
5) The slurry obtained in step (4) was added to the main bulk obtained in step (2) and homogenized.
6) Benzalkonium chloride and Phenyl ethyl alcohol were added to the main bulk obtained in step (2).
7) Weight was made up with Purified water.

### Example 3

| Sr. No. | Ingredients | Qty | |
|---|---|---|---|
| 1. | Ebastine | 100 | mcg/spray |
| 2. | Fluticasone Propionate | 50 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |
| 8. | Disodium edetate | 0.01-0.5% | w/w |

| | | |
|---|---|---|
| 9. | Purified water | q.s. |

### Process;

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Propionate and Ebastine were added to the solution obtained in step (3) to produce uniform slurry.
5) The slurry obtained in step (4) was added to the main bulk obtained in step (2) and homogenized.
6) Benzalkonium chloride and Phenyl ethyl alcohol were added to the main bulk obtained in step (2).
7) Weight was made up with Purified water.

### Example 4

| Sr. No. | Ingredients | Qty | |
|---|---|---|---|
| 1. | Ebastine | 100 | mcg/spray |
| 2. | Fluticasone Propionate | 50 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |

| | | |
|---|---|---|
| 8. | Purified water | q.s. |

Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Propionate and Ebastine were added to the solution obtained in step (3) to produce uniform slurry.
5). The slurry obtained in step (4) was added to the main bulk obtained in step (2) and homogenized.
6) Benzalkonium chloride and Phenyl ethyl alcohol were added to the main bulk obtained in step (2).
7) Weight was made up with Purified water.

### Example 5

| Sr. No. | Ingredients | Qty | |
|---|---|---|---|
| 1. | Ebastine | 200 | mcg/spray |
| 2. | Fluticasone Propionate | 50 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |
| 8. | Disodium edetate | 0.01-0.5% | w/w |

| | | |
|---|---|---|
| 9. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Propionate and Ebastine were added to the solution obtained in step (3) under magnetic stirring to produce uniform slurry.
5) The slurry obtained in step (4) was added to the main bulk obtained in step (2) and homogenized.
6) Benzalkonium chloride, Phenyl ethyl alcohol and Disodium edetate were added to the main bulk obtained in step (2).
7) Weight was made up with Purified water.

### Example 6

| Sr. No. | Ingredients | Qty | |
|---|---|---|---|
| 1. | Ebastine | 200 | mcg/spray |
| 2. | Fluticasone Propionate | 50 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |

| | | |
|---|---|---|
| 8. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Propionate and Ebastine were added to the solution obtained in step (3) under magnetic stirring to produce uniform slurry.
5) The slurry obtained in step (4) was added to the main bulk obtained in step (2) and homogenized.
6) Benzalkonium chloride and Phenyl ethyl alcohol were added to the main bulk obtained in step (2).
7) Weight was made up with Purified water.

### Example 7

| Sr. No. | Ingredients | Qty | |
|---|---|---|---|
| 1. | Ebastine | 1 | mg/spray |
| 2. | Fluticasone Propionate | 50 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |
| 8. | Disodium edetate | 0.01-0.5% | w/w |

| | | |
|---|---|---|
| 9. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Propionate and Ebastine were added to the solution obtained in step (3) to produce uniform slurry.
5) The slurry obtained in step (4) was added to the main bulk obtained in step (2) and homogenized.
6) Benzalkonium chloride, Phenyl ethyl alcohol and Disodium edetate were added to the main bulk obtained in step (2).
7) Weight was made up with Purified water.

### Example 8

| Sr. No. | Ingredients | Qty | |
|---|---|---|---|
| 1. | Ebastine | 1 | mg/spray |
| 2. | Fluticasone Propionate | 50 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |

| | | |
|---|---|---|
| 8. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Propionate and Ebastine were added to the solution obtained in step (3) to produce uniform slurry.
5) The slurry obtained in step (4) was added to the main bulk obtained in step (2) and homogenized.
6) Benzalkonium chloride and Phenyl ethyl alcohol were added to the main bulk obtained in step (2).
7) Weight was made up with Purified water.

### Example 9

| Sr. No. | Ingredients | Qty | |
|---|---|---|---|
| 1. | Ebastine | 2 | mg/spray |
| 2. | Fluticasone Propionate | 50 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |
| 8. | Disodium edetate | 0.01-0.5% | w/w |

| | | |
|---|---|---|
| 9. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Propionate and Ebastine were added to the solution obtained in step (3) to produce uniform slurry.
5) The slurry obtained in step (4) was added to the main bulk obtained in step (2) and homogenized.
6) Benzalkonium chloride, Phenyl ethyl alcohol and Disodium edetate were added to the main bulk obtained in step (2).
7) Weight was made up with Purified water.

### Example 10

| Sr. No. | Ingredients | Qty | |
|---|---|---|---|
| 1. | Ebastine | 2 | mg/spray |
| 2. | Fluticasone Propionate | 50 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |

| | | |
|---|---|---|
| 8. | Purified water | q.s. |

### Process:

1) Anhydrous glucose was added to Purified water
2) Dispersible cellulose was dispersed in filtered Glucose solution obtained from step (1) to produce the main bulk.
3) Polysorbate 80 was dissolved in Purified water.
4) Fluticasone Propionate and Ebastine were added to the solution obtained in step (3) to produce uniform slurry.
5) The slurry obtained in step (4) was added to the main bulk obtained in step (2) and homogenized.
6) Benzalkonium chloride and Phenyl ethyl alcohol were added to the main bulk obtained in step (2).
7) Weight was made up with Purified water.

### Example 11

| Sr. No. | Ingredients | Qty | |
|---|---|---|---|
| 1. | Ebastine | 50 | mcg/spray |
| 2. | Fluticasone Furoate | 27.5 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |
| 8. | Disodium edetate | 0.01-0.5% | w/w |

| | | |
|---|---|---|
| 9. | Purified water | q.s. |

### Process:

1) Anhydrous glucose was added to Purified water
2) Dispersible cellulose was dispersed in filtered Glucose solution obtained from step (1) to produce the main bulk.
3) Polysorbate 80 was dissolved in Purified water.
4) Fluticasone Furoate and Ebastine were added to the solution obtained in step (3) to produce uniform slurry.
5) The slurry obtained in step (4) was added to the main bulk obtained in step (2) and homogenized.
6) Benzalkonium chloride, Phenyl ethyl alcohol and Disodium edetate were added to the main bulk obtained in step (2).
7) Weight was made up with Purified water

### Example 12

| Sr. No. | Ingredients | Qty | |
|---|---|---|---|
| 1. | Ebastine | 50 | mcg/spray |
| 2. | Fluticasone Furoate | 27.5 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |

| | | |
|---|---|---|
| 8. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Furoate and Ebastine were added to the solution obtained in step (3) to produce uniform slurry.
5) The slurry obtained in step (4) was added to the main bulk obtained in step (2) and homogenized.
6) Benzalkonium chloride and Phenyl ethyl alcohol were added to the main bulk obtained in step (2).
7) Weight was made up with Purified water.

### Example 13

| Sr. No. | Ingredients | Qty | |
|---|---|---|---|
| 1. | Ebastine | 100 | mcg/spray |
| 2. | Fluticasone Furoate | 27.5 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |
| 8. | Disodium edetate | 0.01-0.5% | w/w |

| | | |
|---|---|---|
| 9. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Furoate and Ebastine were added to the solution obtained in step (3) to produce uniform slurry.
5) The slurry obtained in step (4) was added to the main bulk obtained in step (2) and homogenized.
6) Benzalkonium chloride, Phenyl ethyl alcohol and Disodium edetate were added to the main bulk obtained in step (2).
7) Volume was made up with Purified water

### Example 14

| Sr. No. | Ingredients | Qty | |
|---|---|---|---|
| 1. | Ebastine | 100 | mcg/spray |
| 2. | Fluticasone Furoate | 27.5 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |

| | | |
|---|---|---|
| 8. | Purified water | q.s. |

Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Furoate and Ebastine were added to the solution obtained in step (3) to produce uniform slurry.
5) The slurry obtained in step (4) was added to the main bulk obtained in step (2) and homogenized.
6) Benzalkonium chloride and Phenyl ethyl alcohol were added to the main bulk obtained in step (2).
7) Weight was made up with Purified water

### Example 15

| Sr. No. | Ingredients | Qty | |
|---|---|---|---|
| 1. | Ebastine | 200 | mcg/spray |
| 2. | Fluticasone Furoate | 27.5 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |
| 8. | Disodium edetate | 0.01-0.5% | w/w |

| | | |
|---|---|---|
| 9. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Furoate and Ebastine were added to the solution obtained in step (3) to produce uniform slurry.
5) The slurry obtained in step (4) was added to the main bulk obtained in step (2) and homogenized.
6) Benzalkonium chloride, Phenyl ethyl alcohol and Disodium edetate were added to the main bulk obtained in step (2).
7) Weight was made up with Purified water

### Example 16

| Sr. No. | Ingredients | Qty | |
|---|---|---|---|
| 1. | Ebastine | 200 | mcg/spray |
| 2. | Fluticasone Furoate | 27.5 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |

| | | |
|---|---|---|
| 8. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Furoate and Ebastine were added to the solution obtained in step (3) under magnetic stirring to produce uniform slurry.
5) The slurry obtained in step (4) was added to the main bulk obtained in step (2) and homogenized.
6) Benzalkonium chloride and Phenyl ethyl alcohol were added to the main bulk obtained in step (2).
7) Weight was made up with Purified water

### Example 17

| Sr. No. | Ingredients | Qty | |
|---|---|---|---|
| 1. | Ebastine | 1 | mg/spray |
| 2. | Fluticasone Furoate | 27.5 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |
| 8. | Disodium edetate | 0.01-0.5% | w/w |

| | | |
|---|---|---|
| 9. | Purified water | q.s. |

### Process:

1) Anhydrous glucose was added to Purified water.
2) Sifted Dispersible cellulose was dispersed in filtered Glucose solution obtained from step (1) to produce the main bulk.
3) Polysorbate 80 was dissolved in Purified water
4) Fluticasone Furoate and Ebastine were added to the solution obtained in step (3) to produce uniform slurry.
5) The slurry obtained in step (4) was added to the main bulk obtained in step (2) and homogenized.
6) Benzalkonium chloride, Phenyl ethyl alcohol and Disodium edetate were added to the main bulk obtained in step (2).
7) Weight was made up with Purified water

### Example 18

| Sr. No. | Ingredients | Qty | |
|---|---|---|---|
| 1. | Ebastine | 1 | mg/spray |
| 2. | Fluticasone Furoate | 27.5 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |

| | | |
|---|---|---|
| 8. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Furoate and Ebastine were added to the solution obtained in step (3) under magnetic stirring to produce uniform slurry.
5) The slurry obtained in step (4) was added to the main bulk obtained in step (2) and homogenized.
6) Benzalkonium chloride and Phenyl ethyl alcohol were added to the main bulk obtained in step (2).
7) Weight was made up with Purified water

### Example 19

| Sr. No. | Ingredients | Qty | |
|---|---|---|---|
| 1. | Ebastine | 2 | mg/spray |
| 2. | Fluticasone Furoate | 27.5 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |
| 8. | Disodium edetate | 0.01-0.5% | w/w |

| | | |
|---|---|---|
| 9. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Furoate and Ebastine were added to the solution obtained in step (3) under magnetic stirring to produce uniform slurry.
5) The slurry obtained in step (4) was added to the main bulk obtained in step (2) and homogenized.
6) Benzalkonium chloride, Phenyl ethyl alcohol and Disodium edetate were added to the main bulk obtained in step (2).
7) Weight was made up with Purified water

### Example 20

| Sr. No. | Ingredients | Qty | |
|---|---|---|---|
| 1. | Ebastine | 2 | mg/spray |
| 2. | Fluticasone Furoate | 27.5 | mcg/spray |
| 3. | Polysorbate 80 | 0.005-0.10% | w/w |
| 4. | Dispersible cellulose | 1.5-2% | w/w |
| 5. | Anhydrous glucose | 5% | w/w |
| 6. | Benzalkonium chloride | 0.02% | w/w |
| 7. | Phenyl ethyl alcohol | 0.25% | w/w |

| | | |
|---|---|---|
| 8. | Purified water | q.s. |

### Process:

1) Anhydrous glucose was added to Purified water.
2) Dispersible cellulose was dispersed in filtered Glucose solution obtained from step (1) to produce the main bulk.
3) Polysorbate 80 was dissolved in Purified water.
4) Fluticasone Furoate and Ebastine were added to the solution obtained in step (3) under magnetic stirring to produce uniform slurry.
5) The slurry obtained in step (4) was added to the main bulk obtained in step (2) and homogenized.
6) Benzalkonium chloride and Phenyl ethyl alcohol were added to the main bulk obtained in step (2).
7) Weight was made up with Purified water

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to fall within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “an excipient” includes a single excipient as well as two or more different excipients, and the like.

1. A pharmaceutical composition comprising at least one antihistamine, at least one corticosteroid, and at least one pharmaceutical excipient, wherein the at least one antihistamine comprises ebastine or its pharmaceutically acceptable salt, solvate, ester or physiologically functional derivative thereof, and wherein the at least one corticosteroid comprises fluticasone or its pharmaceutically acceptable ester thereof.

2. A pharmaceutical composition according to claim 1, wherein the fluticasone comprises fluticasone propionate, fluticasone furoate or fluticasone valerate.

3. A pharmaceutical composition according to claim 1, wherein the fluticasone is present in an amount of from 20 mcg to 50 mcg.

4. A pharmaceutical composition according to claim 1, wherein ebastine is present in an amount of from 25 mcg to 2 g.

5. A pharmaceutical composition according to claim 1, wherein the composition further comprises pharmaceutically acceptable excipients suitable for nasal delivery, and wherein at least one antihistamine and at least one corticosteroid are present in a dosage form suitable for nasal delivery.

6. A pharmaceutical composition according to claim 5, wherein the composition is in the form of a nasal spray, nasal solution, a nasal suspension, a nasal ointment, nasal drops or a nasal gel.

7. A pharmaceutical composition according to claim 5, wherein the pharmaceutically acceptable excipients are selected from the group comprising at least one of pH adjusters, osmotic agents, emulsifiers, dispersing agents, surfactants, solubilizers, buffering agents, preservatives, wetting agents, gelling agents, consistency agents, chelating agents, ciliary stimulants, mucus thinning agents, suspending agents, thickening agents, or combinations thereof.

8. A pharmaceutical composition according to claim 7, wherein the pH adjuster is citric acid, sodium citrate, sodium hydrogen sulphate, borate buffer, sodium hydrogen orthophosphate, disodium hydrogen phosphate, Sodium dihydrogen phosphate, trometamol, acetate buffer, citrate buffer and their hydrous, anhydrous forms or mixtures thereof.

9. A pharmaceutical composition according to claim 7, wherein the osmotic agent is sodium chloride, potassium chloride, zinc chloride, calcium chloride, mannitol, glycerol, and boric acid, citric acid, sodium tartrate, sodium phosphate, potassium phosphate, propylene glycol or other inorganic or organic solutes, dextrose, anhydrous glucose or mixtures thereof.

10. A pharmaceutical composition according to claim 7, wherein the surfactant is an amphoteric, non-ionic, cationic or anionic or combinations thereof.

11. A pharmaceutical composition according to claim 7, wherein the preservative is benzalkonium chloride, benzoic acid or a salt, sodium benzoate, potassium sorbate, sorbic acid or a salt, edetic acid and its alkali salts, lower alkyl phydroxybenzoates, chlorhexidine, phenyl mercury borate, quaternary ammonium compound or mixtures thereof.

12. A pharmaceutical composition according to claim 7, wherein the consistency agents are monosaccharides, disaccharides and other sugars, ribose, glycerine/glycerol, sorbitol, xylitol, inositol, propylene glycol, galactose, mannose, xylose, rhamnose, glutaraldehyde, invert sugars, ethanol, honey, mannitol, polyethylene glycol or mixtures thereof.

13. A pharmaceutical composition according to claim 7, wherein the chelating agent is sodium EDTA or disodium EDTA or mixtures thereof.

14. A pharmaceutical composition according to claim 1, wherein said composition is in the form of nasal spray or nasal drops

15. A pharmaceutical composition according to claim 1, wherein said composition has a pH of from 3.0 to 7.5.

16. A pharmaceutical composition according to claim 1, wherein the composition further comprises pharmaceutically acceptable excipients suitable for ocular delivery, and wherein at least one antihistamine and at least one corticosteroid are present in a dosage form suitable for ocular delivery.

17. A pharmaceutical composition according to claim 16, wherein the composition is in the form of ophthalmic drops, a suspension, a solution, gel, an ointment, in situ gel, occusert or an emulsion.

18. A pharmaceutical composition according to claim 16, wherein the pharmaceutically acceptable excipients comprise at least one of pH adjusters, osmotic agents, emulsifiers, dispersing agents, surfactants, buffering agents, preservatives, wetting agents, gelling agents, consistency agents, chelating agents, suspending agents, thickening agents, or combinations thereof.

19. A pharmaceutical composition according to claim 1, wherein the composition further comprises suitable pharmaceutically acceptable excipients for use in a nasal spray, metered dose inhaler, a dry powder inhaler, a nebuliser or an insufflation powder.

20. A pharmaceutical composition according to any claim 1, wherein the at least one antihistamine and/or at least one corticosteroid are in micronized form.

21. A pharmaceutical composition according to any claim 1, wherein the at least one antihistamine and/or at least one corticosteroid are in nanosize form.

22. A pharmaceutical composition according to claim 1, further comprising at least one or more of an anticholinergic, antiallergic, leukotriene antagonist, decongestant, sympathomimetic agent, mucolytic, opiate analgesic, a lipoxygenase inhibiting compound, or a pharmaceutically acceptable salt, solvate, tautomer, enantiomer, isomer, hydrate, prodrug or polymorph thereof.

23. A metered dose inhaler comprising a pharmaceutical composition according to claim 19.

24. A dry powder inhaler comprising a pharmaceutical composition according to claim 19.

25. A nasal spray comprising a pharmaceutical composition according to claim 19.

26. A container comprising a pharmaceutical composition according to claim 1, preferably wherein the container is adapted for introduction of the pharmaceutical composition to the nasal passages or eyes.

27. A pharmaceutical composition according to claim 1, wherein ebastine, fluticasone and at least one pharmaceutically acceptable excipient are formulated for simultaneous, separate or sequential administration.

28. A pharmaceutical composition according to claim 1 for use in treating disorders or conditions that respond to, or are prevented, ameliorated or eliminated by the administration of an antihistamine and a corticosteroid.

29. A method for the prevention and/or treatment of a disorder or condition that responds to, is prevented, ameliorated or eliminated by the administration of an antihistamine and a corticosteroid, which method comprises administering to a patient in need thereof, a therapeutically effective amount of a composition according to claim 1.

30. A pharmaceutical composition according to claim 28, wherein the disorder or condition is allergic rhinitis.

31. A process for the preparation of a pharmaceutical composition according to claim 1.

32. A process according to claim 31, wherein the at least one antihistamine and/or at least one corticosteroid are in micronized form; or, wherein the at least one antihistamine and/or at least one corticosteroid are in nanosize form; and,
the process comprises any of ball milling, jet milling, sonication, homogenisation or solvent precipitation.

33. A process according to claim 31, wherein the at least one antihistamine and/or at least one corticosteroid are in micronized form; or, wherein the at least one antihistamine and/or at least one corticosteroid are in nanosize form; and
wherein the process comprises any of milling, precipitation, high pressure homogenisation, spray-freeze drying, double emulsion/solvent evaporation, particle replication in non-wetting templates, thermal condensation, or ultrasonication.

34. A process according to claim 31, wherein the pharmaceutical composition further comprises pharmaceutically acceptable excipients suitable for nasal delivery, and wherein at least one antihistamine and at least one corticosteroid are present in a dosage form suitable for nasal delivery, and
wherein the process comprises dissolving at least one corticosteroid and at least one antihistamine, optionally adding other suitable pharmaceutically acceptable excipients, and adjusting the pH using a suitable pH adjusting agent.

35. A process according to claim 31, wherein the process comprises admixing a pharmaceutically acceptable carrier and/or excipient with the at least one corticosteroid and at least one antihistamine and a propellant, and providing the composition in precrimped cans.

36. A process according to claim 31, wherein the process comprises admixing a pharmaceutically acceptable carrier and/or excipient with the at least one corticosteroid and at least one antihistamine and providing the composition as a dry powder inhaler.

37. A method for the treatment of nasal polyps, comprising administering an effective amount of a composition of claim 1 to a subject having nasal polyps.

38. A method for the treatment of urticaria, comprising administering an effective amount of a composition of claim 1 to a subject having urticaria.

39. (canceled)

40. A method according to claim 29, wherein the disorder or condition is allergic rhinitis.

41. A method for the treatment of allergic rhinitis, comprising administering an effective amount of a composition of claim 1 to a subject having allergic rhinitis.
